(19) United States
(12) Patent Application Publication
(10) Pub. No.: US 2020/0048204 A1
(43) Pub. Date: Feb. 13, 2020

(54) CHEMOPROTEOMIC APPROACH FOR DISCOVERING COVALENT LIGANDS FOR DIVERSE PROTEIN TARGETS

(71) Applicant: UNIVERSITY OF SOUTHERN CALIFORNIA, Los Angeles, CA (US)

(72) Inventors: Chao ZHANG, Monterey Park, CA (US); Feng NI, Los Angeles, CA (US); Arunika Ishani EKANAYAKE, Inglewood, CA (US)

(73) Assignee: University of Southern California, Los Angeles, CA (US)

(21) Appl. No.: 16/339,621

(22) PCT Filed: Oct. 4, 2017
(86) PCT No.: PCT/US2017/055074

§ 371 Date: Apr. 4, 2019

Related U.S. Application Data
(60) Provisional application No. 62/403,925, filed on Oct. 4, 2016.

(51) Int. Cl. C07D 215/44; C07D 487/04; C07D 221/10

(52) U.S. Cl. CPC C07D 215/44; C07D 221/10; C07D 487/04

(57) ABSTRACT

Despite its power in identifying highly potent ligands for select protein targets, conventional medicinal chemistry is limited by its low throughput and lack of proteomic selectivity information. We seek to develop a chemoproteomic approach for discovering covalent ligands for protein targets in an unbiased, high-throughput manner. Tripartite probe compounds composed of a heterocyclic core, an electrophilic ‘warhead’ and an alkyne tag have been designed and synthesized for covalently labeling and identifying targets in cells. We have developed a novel condensation reaction to prepare 2-chloromethylquinoline (2-CMQ), a novel electrophilic heterocycle. These chloromethylquinolines potently and covalently bind to a number of cellular protein targets including Prostaglandin E Synthase 2 (PTGES2), a critical regulator of cell proliferation, apoptosis, angiogenesis, inflammation, and immune surveillance.

### CROSS-REFERENCE TO RELATED APPLICATIONS

This application claims the benefit of U.S. provisional application Ser. No. 62/403,925 filed Oct. 4, 2016, the disclosure of which is hereby incorporated in its entirety by reference herein.

### TECHNICAL FIELD

The present disclosure relates to compounds and methods of identifying covalent ligands for protein targets as used in medicinal chemistry.

### BACKGROUND

Availability of various biological assays in high throughput format and access to large chemical libraries have enabled the rapid discovery of small molecules that elicit interesting phenotypes to cells and organisms. However, it is challenging to determine the mechanism of action of the hit compounds from such phenotypic screens. In particular, the cellular targets of the hits are often difficult to identify even with the aid of affinity chromatography, because the complex formed between the small molecule and its protein targets can fall apart during the enrichment and wash steps. The difficulty of target identification has severely limited the therapeutic potential of small molecules with interesting biological activities.

Small molecules that contain electrophilic groups can form covalent linkage with their target proteins, which can be exploited to facilitate the identification of their target proteins. In particular, the covalent modification of proteins at cysteine residues by small molecules has widespread applications in drug discovery. These applications rely on the availability of an arsenal of electrophiles with tunable reactivity and selectivity. Common electrophiles having high selectivity for reaction with the thiol group include halomethylketones, haloacetamides, maleimides, and α,β-unsaturated ketones.

Accordingly, because of the increasing interest in the therapeutic development of electrophilic compounds, there is a strong need for the discovery and preparation of novel electrophiles that exhibit distinct stereo-electronic properties from the ones that are commonly used now.

### SUMMARY

The present invention solves one or more problems of the prior art by providing a compound that is useful in identifying covalent ligands for target proteins. The compound is described by formula I or formula II:

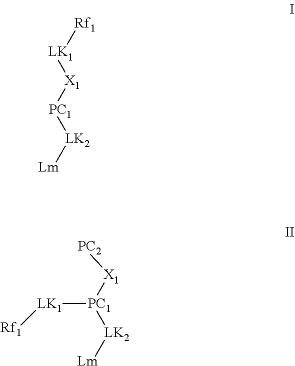

wherein:

Rf₁ is an alkyne-containing moiety or azido-containing moiety;

X₁ is NR or O or X₁ is absent with LK being directly bonded to Pc where R is H or C₁₋₆ alkyl;

LK₁, LK₂ are each independently a hydrocarbon-containing linking group;

PC₁ is aryl or heteroaryl;

PC₂ is an alkyl, aryl, heteroaryl, cycloalkyl, or heteroatom-containing ring system; and

Lm is a leaving group-containing moiety or an electrophilic moiety.

In another embodiment of the present invention, a method of finding covalent ligands is provided. The method comprises contacting a protein-containing sample with any of the above-mentioned compounds to form a modified protein; and contacting the modified protein with a probe compound that includes an alkyne-containing moiety Rf₁ or azido-containing moiety Rf₂, with the proviso that when Rf₁ is an alkyne-containing moiety, Rf₂ is an azido-containing moiety or when Rf₂ is an alkyne-containing moiety, Rf₁ is an azido-containing moiety wherein Rf₁ reacts with Rf₂ to form a ring.

In another embodiment, a method of preparing one class of electrophilic compounds, 2-chloromethylquinolines, is provided. A condensation reaction was discovered to facilitate a single-step conversion of simple starting materials, chloroacetamides, into target molecules, 2-chloromethylquinolines. This method can be readily adapted to parallel synthesis in generating enormous chemical diversity.

In another embodiment, a novel chemoproteomic approach for rapid identification of covalent ligands for target proteins is provided. A library of tripartite probes that contain a heterocyclic core, an electrophilic ‘warhead’ and an alkyne tag, are designed, synthesized and used as a “bait” to covalently catch “prey” proteins in the proteome. This is a rather general approach that can be exercised for any combinations of heterocycles and electrophiles for target-ligand discovery.

In another embodiment, protein targets of 2-chloromethylquinolines are provided. A number of proteins were identified as proteomic targets of 2-chloromethylquinolines with submicromolar or micromolar affinity. They include glutathione S-transferase omega-1, heme oxygenase 2, and prostaglandin E synthase 2, which are involved in human diseases such as cancer, inflammation and immune disorders. These 2-chloromethylquinolines can thus serve as lead compounds for the development of novel therapeutics by modulating the activity of these target proteins under disease settings.

### BRIEF DESCRIPTION OF THE DRAWINGS

FIGS. 1A-B. A novel approach for proteome-wide discovery of covalent ligands is shown. FIG. 1A shows an analogy between tri-partite small-molecule probes and fishing gears. FIG. 1B shows a schematic representation of the procedure for proteome-wide discovery of covalent ligands using tri-partite probes to fish out protein targets from proteomes.

FIGS. 2A-B. A novel one-step condensation reaction yielding a panel of diverse 2-chloromethylquinoline (2-CMQ) probes is shown. FIG. 2A shows a condensation reaction developed from simple acetamides to access 2-CMQs, a class of novel electrophilic heterocyclic compounds. FIG. 2B shows a panel of synthesized 2-CMQ probes.

FIGS. 3A-C. Proteome labeling by 2-chloromethylquinoline probes in situ is shown. FIG. 3A shows in-gel fluorescence images of proteome labeled by eight 2-CMQ probes. HEK293H cells were treated with the probe at indicated concentrations, lysed, clicked with TAMRA-azide, and resolved by SDS-PAGE before being fluorescence imaged. Bands A-F denote the major targets that are labeled by compound 16. FIG. 3B shows that coomassie staining of the same gel shows largely even loading across the lanes.

FIG. 4. A pulse-chase experiment revealed apparent affinity of 2-CMQ to its targets in situ. Cells were pretreated with varying concentrations of compound 17 that contains no alkyne tag for 1 hr followed by incubation of 3 μM of probe 3 for 1 hr. Cells were then lysed, clicked with TAMRA-azide, and resolved by SDS-PAGE before being fluorescence imaged.

FIGS. 5A-B. Targets of 2-CMQs were identified using affinity pull-down followed by LC-MS/MS analysis. FIG. 5A shows target proteins enriched and visualized by coomassie staining. Cells were treated with probe 15, clicked to biotin-azo-azide, and enriched on streptavidin resin. The target proteins were then eluted with sodium dithionite, resolved by SDS-PAGE, and stained with coomassie blue. Slabs were cut off the gel for trypsinization and LC-MS/MS analysis. FIG. 5B shows three proteins that were identified from the gel slabs by LC-MS/MS.

FIG. 6. Confirmation of PTGES2 as the target responsible for band D and identification of the probe modification site. Transient transfection of FLAG-tagged wild-type PTGES2 soluble fragment led to enhanced fluorescent band at ˜37 kDa. When a PTGES2 variant in which Cys110 was mutated to Ala was used instead, no fluorescence increase was observed. The FLAG western blot revealed comparable levels of PTGES2 proteins across the transfection conditions.

FIG. 7. Recombinant PTGES2 was labeled by probe 15 near saturation at low micromolar concentrations. 6×His-tagged PTGES2 was expressed and affinity-purified from E. coli. The protein was incubated with probe 15 at 37° C. for 1 hr, followed by click reaction with TAMRA-azide and fluorescence imaging.

FIG. 8. Recombinant PTGES2 was occupied by compound 17 at low micromolar concentrations. 6×His-tagged PTGES2 was expressed and affinity-purified from E. coli. The protein was first treated with various concentrations of compound 17 at 37° C. for 1 hr, then incubated with 3 μM of probe 15 at 37° C. for 1 hr, followed by click reaction with TAMRA-azide and fluorescence imaging.

### DETAILED DESCRIPTION

Reference will now be made in detail to presently preferred compositions, embodiments and methods of the present invention, which constitute the best modes of practicing the invention presently known to the inventors. The Figures are not necessarily to scale. However, it is to be understood that the disclosed embodiments are merely exemplary of the invention that may be embodied in various and alternative forms. Therefore, specific details disclosed herein are not to be interpreted as limiting, but merely as a representative basis for any aspect of the invention and/or as a representative basis for teaching one skilled in the art to variously employ the present invention.

Except in the examples, or where otherwise expressly indicated, all numerical quantities in this description indicating amounts of material or conditions of reaction and/or use are to be understood as modified by the word “about” in describing the broadest scope of the invention. Practice within the numerical limits stated is generally preferred. Also, unless expressly stated to the contrary: all R groups (e.g. Rᵢ where i is an integer) include alkyl, lower alkyl, C₁₋₆ alkyl, C₆₋₁₀ aryl, or C₆₋₁₀ heteroaryl; single letters (e.g., “n” or “o”) are 1, 2, 3, 4, or 5; percent, “parts of,” and ratio values are by weight; the term “polymer” includes “oligomer,” “copolymer,” “terpolymer,” and the like; molecular weights provided for any polymers refers to weight average molecular weight unless otherwise indicated; the description of a group or class of materials as suitable or preferred for a given purpose in connection with the invention implies that mixtures of any two or more of the members of the group or class are equally suitable or preferred; description of constituents in chemical terms refers to the constituents at the time of addition to any combination specified in the description, and does not necessarily preclude chemical interactions among the constituents of a mixture once mixed; the first definition of an acronym or other abbreviation applies to all subsequent uses herein of the same abbreviation and applies mutatis mutandis to normal grammatical variations of the initially defined abbreviation; and, unless expressly stated to the contrary, measurement of a property is determined by the same technique as previously or later referenced for the same property.

It is also to be understood that this invention is not limited to the specific embodiments and methods described below, as specific components and/or conditions may, of course, vary. Furthermore, the terminology used herein is used only for the purpose of describing particular embodiments of the present invention and is not intended to be limiting in any way.

It must also be noted that, as used in the specification and the appended claims, the singular form “a,” “an,” and “the” comprise plural referents unless the context clearly indicates otherwise. For example, reference to a component in the singular is intended to comprise a plurality of components.

The term “comprising” is synonymous with “including,” “having,” “containing,” or “characterized by.” These terms are inclusive and open-ended and do not exclude additional, unrecited elements or method steps.

The phrase “consisting of” excludes any element, step, or ingredient not specified in the claim. When this phrase appears in a clause of the body of a claim, rather than immediately following the preamble, it limits only the element set forth in that clause; other elements are not excluded from the claim as a whole.

The phrase “consisting essentially of” limits the scope of a claim to the specified materials or steps, plus those that do not materially affect the basic and novel characteristic(s) of the claimed subject matter.

With respect to the terms “comprising,” “consisting of,” and “consisting essentially of,” where one of these three terms is used herein, the presently disclosed and claimed subject matter can include the use of either of the other two terms.

As used herein “alkyl” refers to C₁₋₂₀ inclusive, linear (i.e., “straight-chain”), branched, saturated or at least partially and in some cases fully unsaturated (i.e., alkenyl and alkynyl) hydrocarbon chains, including for example, methyl, ethyl, propyl, isopropyl, butyl, isobutyl, tert-butyl, pentyl, hexyl, octyl, ethenyl, propenyl, butenyl, pentenyl, hexenyl, octenyl, butadienyl, propynyl, butynyl, pentynyl, hexynyl, heptynyl, and allenyl groups. “Branched” refers to an alkyl group in which a lower alkyl group, such as methyl, ethyl or propyl, is attached to a linear alkyl chain. “Lower alkyl” refers to an alkyl group having 1 to about 8 carbon atoms (i.e., a C₁₋₈ alkyl), e.g., 1, 2, 3, 4, 5, 6, 7, or 8 carbon atoms. “Higher alkyl” refers to an alkyl group having about 10 to about 20 carbon atoms, e.g., 10, 11, 12, 13, 14, 15, 16, 17, 18, 19, or 20 carbon atoms. The alkyl group can be optionally substituted (i.e., a “substituted alkyl”) with another atom or functional group such as alkyl, substituted alkyl, halogen, aryl, substituted aryl, alkoxyl, hydroxyl, nitro, amino, alkylamino, dialkylamino, sulfate, mercapto, and the like.

As used herein “alkenyl” means a substituted or unsubstituted monovalent unsaturated hydrocarbon group that has at least one carbon to carbon double bond. The alkenyl can be linear or branched. In a refinement, the alkenyl can have 1, 2 or 3, carbon-carbon double bonds. In a refinement, the alkenyl groups have from 2 to 10 carbon atoms. Examples of alkenyl groups include, but are not limited to, ethenyl, n-propenyl, isopropenyl, n-but-2-enyl, n-hex-3-enyl and the like. The term “alkenylene” refers a divalent alkenyl group.

As used herein “alkynyl” means a monovalent unsaturated hydrocarbon group which may be linear or branched and which has a carbon-carbon triple bond. In a refinement, such alkynyl groups have from 2 to 10 carbon atoms. Representative alkynyl groups include, by way of example, ethynyl, n-propynyl, n-but-2-ynyl, n-hex-3-ynyl and the like. The term “alkynylene” means a divalent alkynyl group. Therefore, the term alkyne-containing moiety includes C₂₋₁₀ alkynyl groups or C₂₋₁₀ alkynylene groups. In context of the present invention, alkyne-containing moiety preferably have one carbon-carbon triple bond.

As used herein “aryl” means a monovalent aromatic hydrocarbon having a single ring (i.e., phenyl) or fused rings (i.e., naphthalene). In a refinement, such aryl groups include from 6 to 12 carbon ring atoms. In another refinement, such aryl groups include 6 to 10 carbon ring atoms. Representative aryl groups include, by way of example, phenyl biphenyl, naphthyl, anthranyl, and naphthalene-1-yl, naphthalene-2-yl, and the like. The term “arylene” means a divalent aryl group. Each aryl can be substituted with a functional group such as alkyl, substituted alkyl, halogen, aryl, substituted aryl, alkoxyl, hydroxyl, nitro, amino, alkylamino, dialkylamino, sulfate, mercapto, and the like.

As used herein “heteroaryl” means a monovalent aromatic group having a single ring or two fused rings and containing in the ring at least one heteroatom (typically 1 to 3 heteroatoms) selected from nitrogen, oxygen or sulfur. In a refinement, heteroaryl groups typically contain from 5 to 10 total ring atoms. In a refinement, heteroaryl groups have from 6 to 16 total ring atoms. In a refinement, the heteroaryl is a C₅₋₁₂ heteroaryl. Examples of heteroaryl include, but are not limited to, monovalent species of pyrrole, imidazole, thiazole, oxazole, furan, thiophene, triazole, pyrazole, isoxazole, isothiazole, pyridine, pyrazine, pyridazine, pyrimidine, triazine, indole, benzofuran, benzothiophene, benzimidazole, benzthiazole, quinoline, isoquinoline, quinazoline, quinoxaline and the like, where the point of attachment is at any available carbon or nitrogen ring atom. Additional examples heteroaryl groups include, but are not limited to, furanyl, thienyl, and pridinyl group. The term “heteroarylene” means a divalent heteroaryl group. Each heteroaryl can be substituted with a functional group such as alkyl, substituted alkyl, halogen, aryl, substituted aryl, alkoxyl, hydroxyl, nitro, amino, alkylamino, dialkylamino, sulfate, mercapto, and the like.

As used herein, “polycyclic heteroaryl” means heteroaryl groups having at least 2 total rings (e.g., fused rings). In a refinement, polycyclic heteroaryl groups have from 9 to 18 total ring atoms. In another refinement, polycyclic heteroaryl groups have from 9 to 15 total ring atoms. In some refinements, the polycyclic heteroaryl groups are C₆₋₁₆ polycyclic heteroaryl groups.

As used herein “alkyl aryl” means a substituted or unsubstituted functional group that includes an aromatic ring (e.g., phenyl, biphenyl, naphthyl, etc) with an attached C₁₋₈ alkyl group.

As used herein “alkyl heteroaryl” means a substituted or unsubstituted heteroaryl group (typically, C₄₋₁₅) with an attached C₁₋₈ alkyl group.

As used herein “heteroatom-containing ring system” means a substituted or unsubstituted functional group that includes aromatic or aliphatic ring systems containing 1 to 3 heteroatoms such as nitrogen, sulfur, and oxygen. In a refinement, the heteroatom-containing ring system has from 5 to 10 total ring atoms. In a refinement, heteroaryl groups have from 6 to 16 total ring atoms. In a refinement, the heteroatom-containing ring system is a C₅₋₁₂ heteroatom-containing ring system.

As used herein “leaving group” means a functional group or atom which can be displaced by another functional group or atom in a substitution reaction (e.g., a nucleophilic substitution reaction). Examples of leaving groups include, but are not limited to, chloro, bromo and iodo groups; sulfonic ester groups (e.g., tosylate, mesylate, brosylate, nosylate, etc.); and acyloxy groups (e.g., trifluoroacetoxy, etc).

Throughout this application, when a group is described as substituted, examples of substituents include, but are not limited to, nitro, cyano, halo (e.g., F, Cl, Br, I), hydroxyl, ester, carboxylate, and the like.

In an embodiment of the present invention, a compound that is useful for identifying covalent ligands is provided. The compound of this embodiment is described by formula I or formula II:

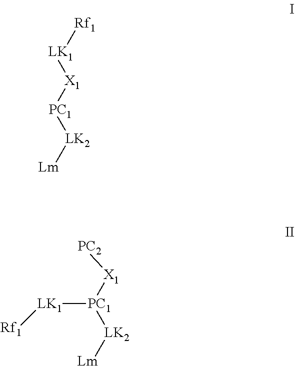

wherein:

Rf₁ is an alkyne-containing moiety (e.g., an ethynyl-containing moiety) or azido-containing moiety;

X₁ is NR or O or X₁ is absent with LK being directly bonded to Pc where R is H or C₁₋₆ alkyl;

LK₁, LK₂ are each independently a hydrocarbon-containing linking group;

PC₁ is aryl or heteroaryl;

PC₂ is an alkyl, aryl, heteroaryl, cycloalkyl, or heteroatom-containing ring system; and

Lm is a leaving group-containing moiety or an electrophilic moiety.

In a refinement of the present embodiment, PC₁ and/or PC2 are each independently a C₆₋₁₂ aryl, or C₅₋₁₂ heteroaryl. In another refinement, PC₁ and/or PC2 are each independently a polycyclic heteroaryl (e.g., a C₇₋₁₂ polycyclic heteroaryl). In another refinement of the present embodiment, PC₁ and/or PC2 are each independently:

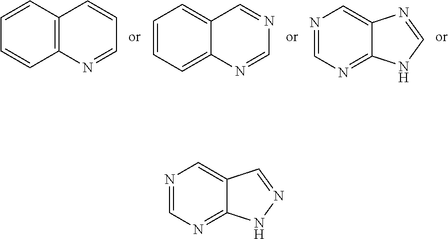

where hydrogens are replaced with Lm, Lk₁, Lk₂, and/or Rf₁.

In some variations, LK₁ or LK₂ are each independently a substituted or unsubstituted alkyl, a substituted or unsubstituted aryl, a substituted or unsubstituted heteroaryl. In a refinement, LK₁ or LK₂ are each independently a substituted or unsubstituted C₁₋₁₂ alkyl, substituted or unsubstituted C₆₋₁₂ aryl, or substituted or unsubstituted C₅₋₁₂ heteroaryl.

In most variations, Lm is Cl, CH═CH₂, or N═C═S. In a refinement, Rf₁ is:

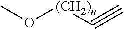

where n is 1-6 and R₁ is H or a C₁₋₆ alkyl. In another variation, Rf₁ is N₃ or

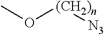

and n is 1-6. Lm can be halo or CH(X₂)R₁ where X₂ is a leaving group and R₁ is H or C₁₋₆ alkyl.

In a variation of the present embodiment, the compound that is useful for identifying covalent ligands is described by formula III:

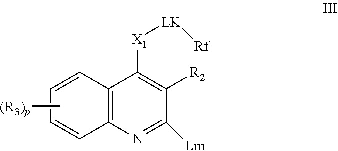

wherein:

R₂ is H, halo, C₁₋₆ alkyl, nitro, or cyano;

R₃ is halo, C₁₋₆ alkyl, nitro, cyano or aryl; and

p is 0, 1, 2, 3, or 4.

In another variation of the present embodiment, the compound that is useful for identifying covalent ligands is described by formula IV:

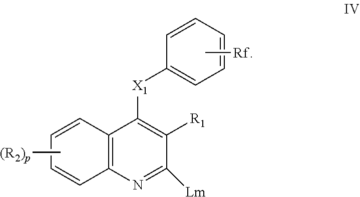

In another variation of the compound that is useful for identifying covalent ligands, is described by formula V:

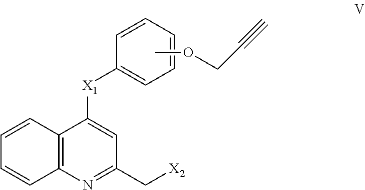

wherein X₂ is a leaving group or an electrophilic functional group.

In another variation of the compound, that is useful for identifying covalent ligands, is described by formula VI:

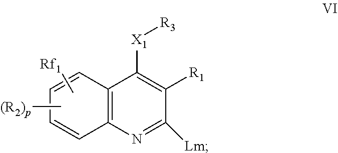

wherein R₂ is halo, C₁₋₆ alkyl, nitro, or cyano;

R₃ is C₁₋₁₂ alkyl, C₆₋₁₂ aryl, C₅₋₁₂ heteroaryl, C₂₋₂₄ alkyl ether groups, C₁₂₋₂₄ aryl ether groups, or C₁₂₋₂₄ aryl alkyl ether groups; and

p is 0, 1, 2, or 3.

In a refinement, Rf₁ is

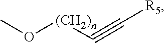

n is 1-6, and R₅ is H or C₁₋₆ alkyl. In another refinement of the present embodiment, Rf₁ is N₃ or

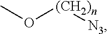

and n is 1-6.

In a variation of the present embodiment, the compound that is useful for identifying covalent ligands is described by formula VII:

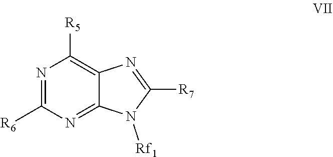

wherein R₅, R₆, R₇ are each independently halo, C₁₋₆ alkyl, nitro, or cyano.

In a variation of the present embodiment, the compound is described by formula VIII:

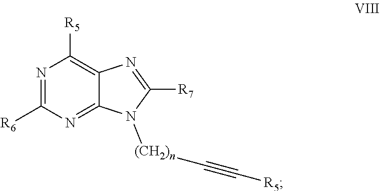

wherein:

n is 1-6; and

R₅ is H or C₁₋₆ alkyl.

In another variation of the compound, that is useful for identifying covalent ligands, is described by formula IX:

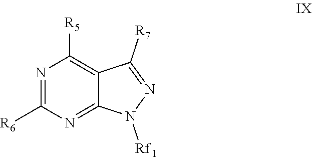

wherein R₅, R₆, R₇ are each independently halo, C₁₋₆ alkyl, nitro, or cyano.

In a variation of the present embodiment, the compound that is useful for identifying covalent ligands is described by formula X:

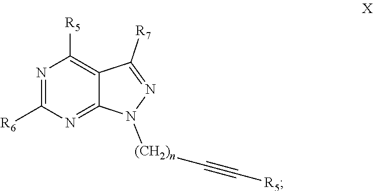

wherein:

n is 1-6; and

R₅ is H or C₁₋₆ alkyl.

In another embodiment of the present invention, a method of finding covalent ligands is provided. The method comprises contacting a protein-containing sample with any of the above-mentioned compounds to form a modified protein; and contacting the modified protein with a probe compound that includes an alkyne-containing moiety Rf₁ or azido-containing moiety Rf₂, with the proviso that when Rf₁ is an alkyne-containing moiety, Rf₂ is an azido-containing moiety or when Rf₂ is an alkyne-containing moiety, Rf₁ is an azido-containing moiety wherein Rf₁ reacts with Rf₂ to form a ring:

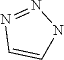

In a refinement of the method of finding covalent ligands, the compound includes a fluorophore.

The following examples illustrate the various embodiments of the present invention. Those skilled in the art will recognize many variations that are within the spirit of the present invention and scope of the claims.

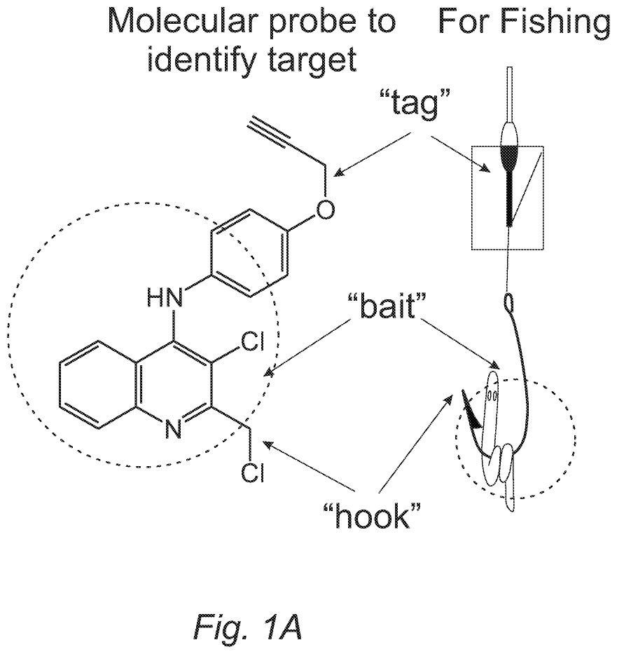

Quinolines and their derivatives are one of the most important classes of heterocyclic compounds. According to the DrugBank, 47 FDA-approved drugs contain a quinoline core. These quinoline-containing drugs exhibit a broad range of medicinal activities including antimalarial, antibacterial, antihypertensive, anti-HIV, antitubercular, anticancer, and antiasthmatic activity. The quinoline thus represents a “privileged” pharmacophore in medicine. 2-Chloromethylquinolines (2-CMQ) contain an electrophile of chloromethyl group attached at the 2 position of the quinoline ring. Herein, we developed a novel condensation reaction to synthesize a panel of tripartite 2-CMQs that contain a quinoline core, an electrophilic ‘warhead’ and an alkyne reporter. These 2-CMQs were evaluated for their covalent interactions with the whole proteome. The alkyne tag facilitated the use of copper-assisted azide-alkyne cycloaddition (CuAAC) for in-gel fluorescence analysis and mass-spectrometry-based identification of protein targets (FIG. 1).

A limited number of methods were reported for the preparation of 2-CMQ in the past. For example, 2-CMQs can be synthesized from quinaldines by converting them to their N-oxides, followed by the reaction with sulfonyl chlorides. A second method is the radical-mediated chlorination reaction of 2-methylquinolines with N-chlorosuccinimide. However, this method suffers from the formation of multiple side products including dichlorinated, trichlorinated, and other undesired side products. Chlorination of 2-methylquinolines using chlorine as the halogen source has benefits of low cost and high yield, but chlorine is an irritating toxic gas. Although 2-CMQ can be prepared from 2-quinolinemethanols by an alternative chlorination approach, the starting materials 2-quinolinemethanols are usually not commercially available. Chlorination of 2-methylquinolines into 2-(chloromethyl)quinolines in the tetrabutylammonium iodide and 1,2-dichloroethane system, via in situ generated ICl, has been developed recently. Unfortunately, all of the above methods are either incompatible with alkyne functional group or require multi-step synthesis to produce the tripartite quinolines (FIG. 1A). A more efficient method is needed to generate probes of diverse functionality and substitution pattern for interacting with various targets in the proteome.

To fill in the gap, we have developed a one-step condensation method for the preparation of 2-chloromethylquinolines. In this approach, PCl₅ is used to condense two molecules of arylacetamide, a readily available material with tremendous choices of structural diversity, to construct the quinoline core, and meantime installs both chloromethyl electrophile and alkyne tag. By changing the substitution position and pattern, we can attach the alkyne tag to various positions in the N-arylacetamide and alter geometries in the probes. In a similar manner, a non-alkyne substituent can also be introduced to broaden the diversity and functionality in the probes.

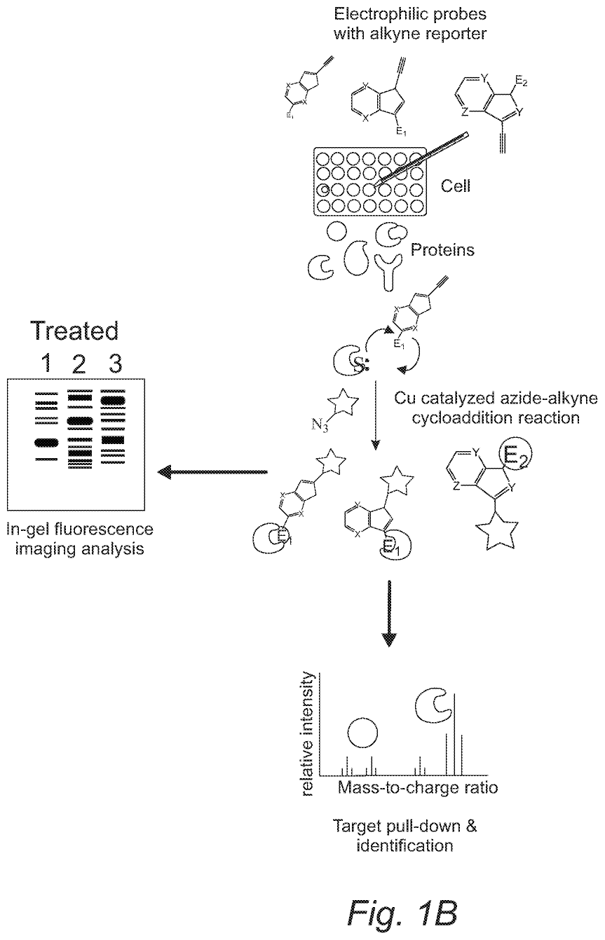

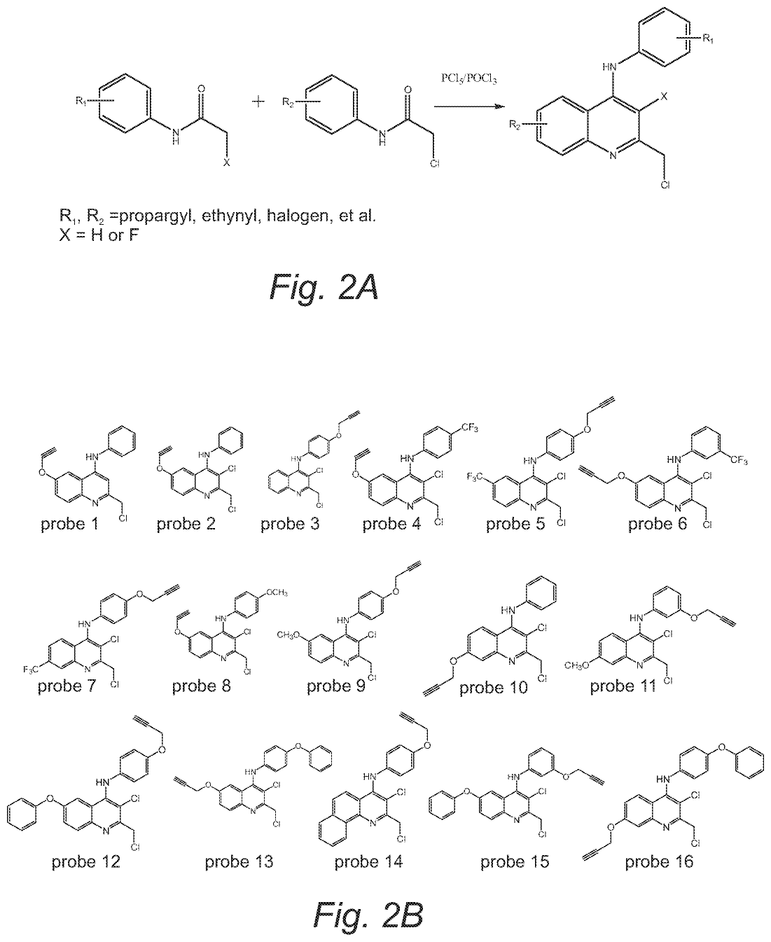

We first examined the proteome reactivity of a panel of 2-CMQs (FIG. 2B). HEK293H cells were treated with each probe at 0.1 or 1.0 μM for 1 hour before being lysed and clicked with TAMRA-azide using CuAAC. The samples were resolved by SDS-PAGE and then scanned for in-gel fluorescence (FIG. 3, lanes selected). 2-CMQs potently labeled a few distinct bands in the proteome. For example, 1 μM of probe 16 labeled six prominent bands of sizes between 20 and 60 kDa (FIG. 3A). The different probes have subtly different labeling patterns indicating different spectrum of protein targets. These data demonstrate that the proteome reactivity of 2-CMQs can be finely adjusted by modulating the sterics of the quinoline ring system, thereby providing a highly tunable electrophile for chemoproteomic studies.

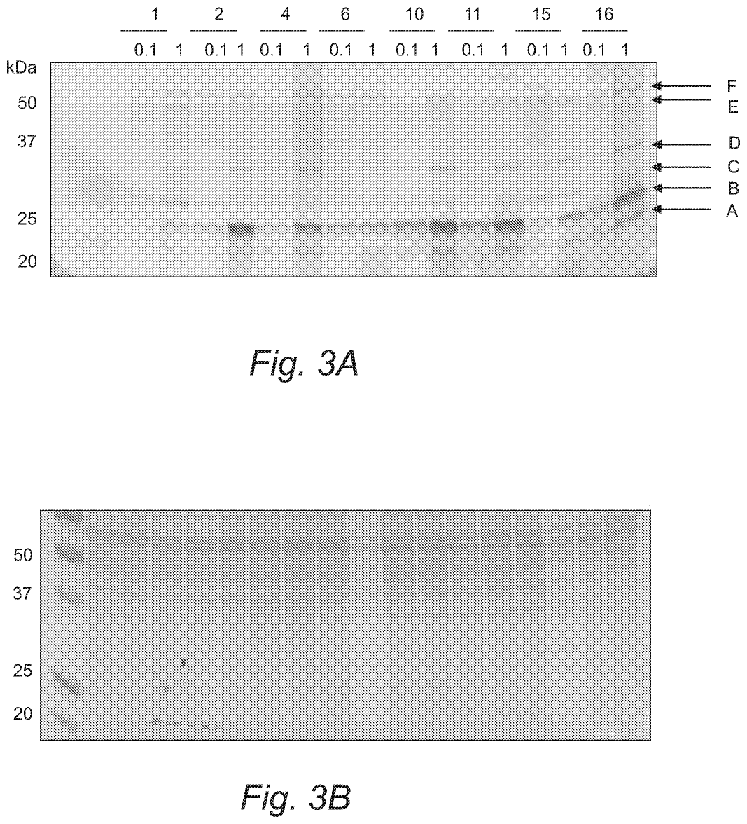

We then chose probe 6 and probe 15 as exemplary probes for further investigation of their proteomic targets. These two probes produced different labeling patterns as band C was labeled by probe 15 but not probe 6 (FIG. 4). To determine the binding saturability and affinity, we prepared two compounds 6c and 15c (FIG. 4) that contain a methoxy group instead of the propargoxy group using the same condensation reaction. The pulse-chase experiment involved pretreating cells with various concentrations of a competitor for 1 hr followed by incubation of 3 μM of a probe for 1 hr. Cells were then lysed, clicked with TAMRA-azide, and resolved by SDS-PAGE before being fluorescence imaged. The competition assay showed that majority of the band labelings were wiped off by competitor compounds, supporting that the labeling is saturable. IC₅₀ values derived from the dose-response curve signify apparent binding affinity of the competitor to the target protein in situ. Our results revealed IC₅₀ values of ˜1 μM for binding of the 2-CMQs to targets A-D, a good starting point considering that no chemical optimization was done yet.

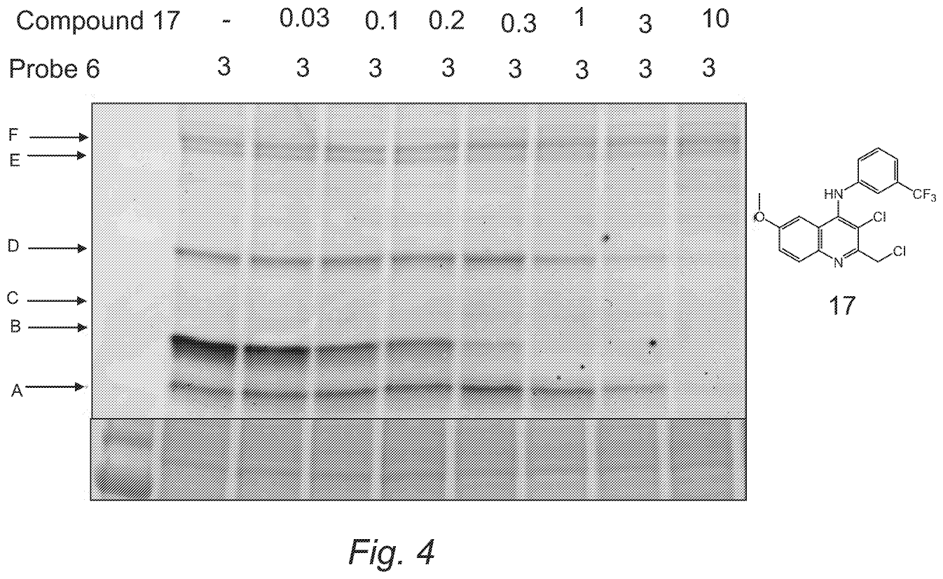

We then set out to identify the target proteins that were covalently labeled by the 2-CMQ probes. HEK293H cells were incubated with mock or 10 μM of 15c for 1 hr before further incubation with 3 μM of probe 15 for 1 more hr at 37° C. The cells were then lysed, conjugated with biotin-azo-azide via CuAAC, and pulled down using streptavidin agarose beads. Addition of sodium dithionite cleaved the target proteins off the beads, which were resolved by SDS-PAGAE and visualized by coomassie staining (FIG. 5). The sections corresponding to the region containing bands A-D were cut off from the gel and subjected to MS analysis. This led to the identification of glutathione S-transferase omega-1, heme oxygenase 2, and prostaglandin E synthase 2 as target proteins B, C and D, respectively.

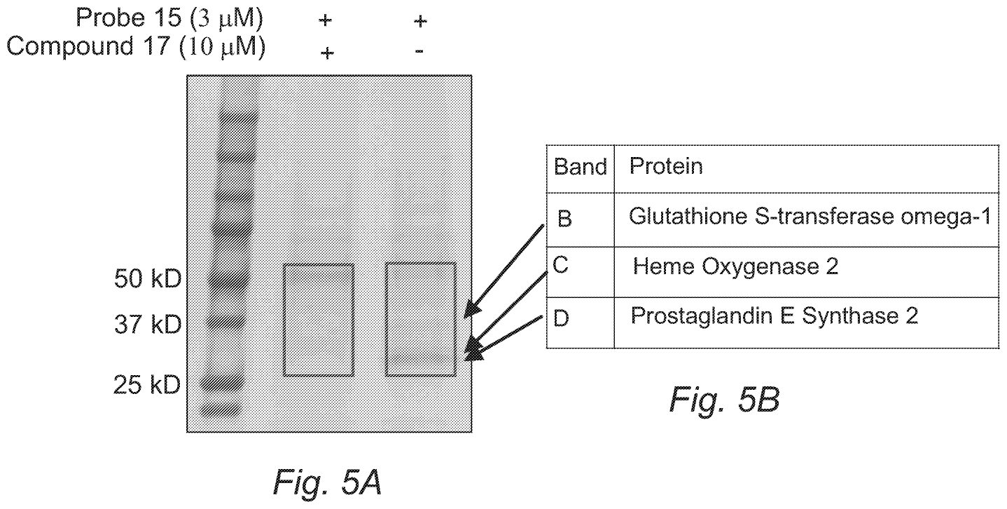

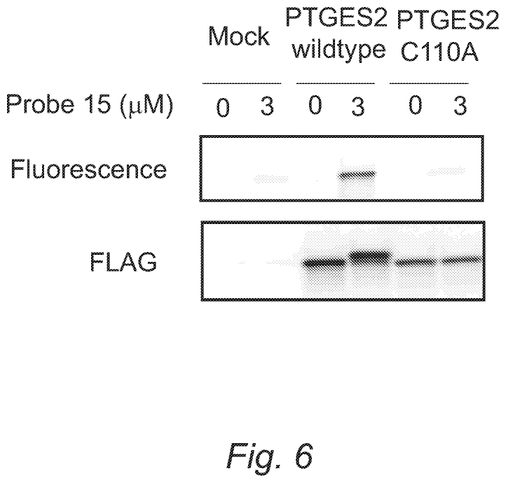

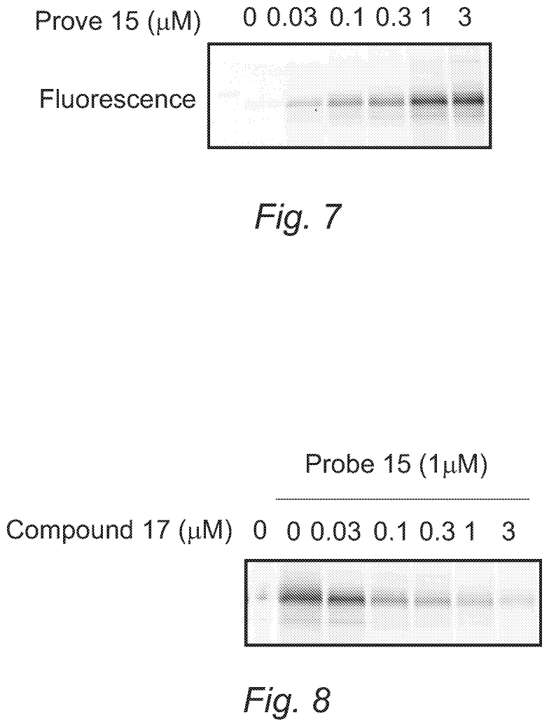

Transient transfection of FLAG-tagged wild-type PTGES2 soluble fragment led to enhanced fluorescent band at ˜37 kDa, confirming PTGES2 as the target protein responsible for band D (FIG. 6). When a PTGES2 variant in which Cys110 was mutated to Ala was transfected instead, no fluorescence increase was observed supporting that Cys110 is the site of probe modification (FIG. 6). To test if pure PTGES2 could also be labeled by 2-CMQ, 6×His-tagged PTGES2 was expressed and affinity-purified from E. coli. The recombinant PTGES2 was labeled by low micromolar of probe 15 to saturated levels, exhibiting similar behaviors to the protein in HEK293H cells (FIG. 7). In addition, pretreatment of the recombinant PTGES2 with increasing concentrations of compound 17 blocked the protein from subsequent labeling by probe 15, suggesting that 2-CMQ2 binding to PTGES2 is close to saturation at 1 μM (FIG. 8).

In conclusion, we have developed a novel condensation reaction to prepare 2-chloromethylquinoline, a new electrophile for chemoproteomic studies. These chloromethylquinolines covalently and potently bind to a number of cellular protein targets including Prostaglandin E Synthase 2, a critical regulator of cell proliferation, apoptosis, angiogenesis, inflammation, and immune surveillance. The novel PTGES2 inhibitors that we discovered using chemoproteomics have the potential to serve as novel therapies for the treatment of human diseases such as inflammation. Beyond this case study, our methodology can be extended to other electrophiles and scaffolds for discovering covalent ligands for diverse targets in the whole proteome.

While exemplary embodiments are described above, it is not intended that these embodiments describe all possible forms of the invention. Rather, the words used in the specification are words of description rather than limitation, and it is understood that various changes may be made without departing from the spirit and scope of the invention. Additionally, the features of various implementing embodiments may be combined to form further embodiments of the invention.

1. A compound having formula I or formula II: 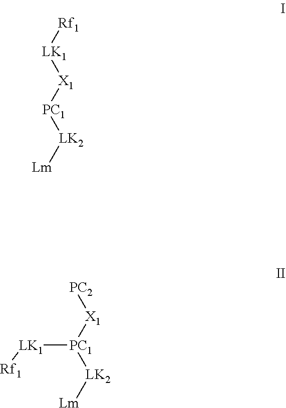
wherein:
Rf₁ is an alkyne-containing moiety or azido-containing moiety;
X₁ is NR or O or X₁ is absent with LK being directly bonded to Pc where R is H or C₁₋₆ alkyl;
LK₁, LK₂ are each independently a hydrocarbon-containing linking group;
PC₁ is aryl or heteroaryl;
PC₂ is an alkyl, aryl, heteroaryl, cycloalkyl, or heteroatom-containing ring system; and
Lm is a leaving group-containing moiety or an electrophilic moiety

2. The compound of claim 1 wherein PC₁ is C₆₋₁₂ aryl, or C₅₋₁₂ heteroaryl.

3. The compound of claim 1 wherein PC₂ is: 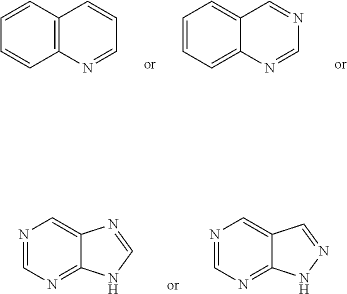 [SMILES: c1ccc2ncccc2c1 ; c1ccc2ncncc2c1 ; c1ncc2nc[nH]c2n1 ; c1ncc2cn[nH]c2n1]
where hydrogens are replaced with Lm and Lk or Rf₁.

4. The compound of claim 1 wherein LK₁ or LK₂ is C₁₋₁₂ alkyl, C₆₋₁₂ aryl, or C₅₋₁₂ heteroaryl.

5. The compound of claim 1 wherein Lm is Cl, CH═CH₂, or N═C═S.

6. The compound of claim 1 wherein:
Rf₁ is 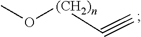
n is 1-6; and
R₁ is H or C₁₋₆ alkyl.

7. The compound of claim 1 wherein:
Rf₁ is N₃ or 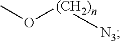
and
n is 1-6.

8. The compound of claim 1 wherein:
Lm is halo or CH(X₂)R₁;
X₂ is a leaving group; and
R₁ is H or C₁₋₆ alkyl.

9. The compound of claim 1 having formula III: 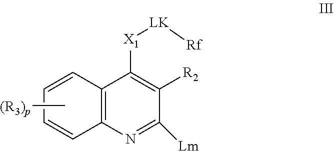
wherein:
R₂ is H, halo, C₁₋₆ alkyl, nitro, or cyano;
R₃ is halo, C₁₋₆ alkyl, nitro, cyano or aryl; and
p is 0, 1, 2, 3, or 4.

10. The compound of claim 9 having formula IV:

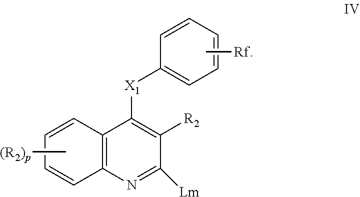

11. The compound of claim 10 having formula V: 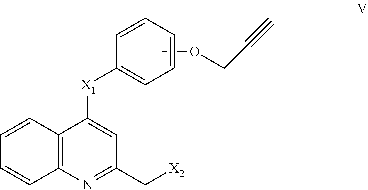
wherein X₂ is a leaving group or an electrophilic functional group.

12. The compound of claim 1 having formula VI: 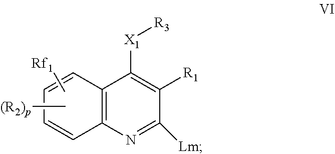
wherein R₂ is halo, C₁₋₆ alkyl, nitro, or cyano;
R₃ is C₁₋₁₂ alkyl, C₆₋₁₂ aryl, C₅₋₁₂ heteroaryl, C₂₋₂₄ alkyl ether groups, C₁₂₋₂₄ aryl ether groups, or C₁₂₋₂₄ aryl alkyl ether groups; and
p is 0, 1, 2, or 3.

13. The compound of claim 12 wherein:
Rf₁ is 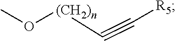
n is 1-6; and
R₅ is H or C₁₋₆ alkyl.

14. The compound of claim 1 wherein:
Rf₁ is N₃ or 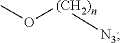
and
n is 1-6.

15. The compound of claim 1 having formula VII: 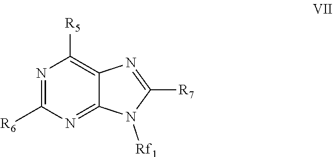
wherein R₅, R₆, R₇ is halo, C₁₋₆ alkyl, nitro, or cyano.

16. The compound of claim 15 having formula VIII: 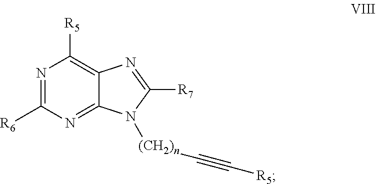
wherein:
n is 1-6; and
R₅ is H or C₁₋₆ alkyl.

17. The compound of claim 1 having formula IX: 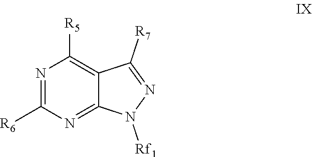
R₅, R₆, R₇ is halo, C₁₋₆ alkyl, nitro, or cyano.

18. The compound of claim 17 having formula X: 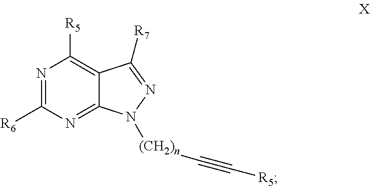
wherein:
n is 1-6; and
R₅ is H or C₁₋₆ alkyl.

19. A method of finding covalent ligands comprising:
contacting a protein-containing sample with any of the compounds of claim 1 to form a modified protein; and
contacting the modified protein with a probe compound that includes an alkyne-containing moiety Rf₁ or azido-containing moiety Rf₂, with the proviso that when Rf₁ is an alkyne-containing moiety, Rf₂ is an azido-containing moiety or when Rf₂ is an alkyne-containing moiety, Rf₁ is an azido-containing moiety wherein Rf₁ reacts with Rf₂ to form a ring.

20. The method of claim 19 wherein the probe compound includes a fluorophore.
